(19) United States
(12) Patent Application Publication
(10) Pub. No.: US 2002/0177223 A1
(43) Pub. Date: Nov. 28, 2002

(54) Methods and compositions for crosslinking tissue

(76) Inventors: Mathew F. Ogle, St. Paul, MN (US); M. William Mirsch II, Roseville, MN (US)

Correspondence Address: Peter S. Dardi, Patterson, Thuente, Skaar & Christensen, P.A., 4800 IDS Center, 80 South 8th Street, Minneapolis, MN, 55402-2100, US

(21) Appl. No.: 09/804,408

(22) Filed: Mar. 12, 2001

(51) Int. Cl. C12N 5/06

(52) U.S. Cl. 435/325

(57) ABSTRACT

Tissue is crosslinked with compositions that include bridges. In some embodiments, the tissue includes linkers that bond to the tissue and bridges that connect two or more of the linkers. In other embodiments, bridges connect two or more modified sites in a modified tissue. The use of bridges in the crosslinking methods more readily forms crosslinks, particularly between different polypeptides in the tissue. The use of bridges can allow for greater modulation of the degree of crosslinking of a tissue.

### BACKGROUND OF THE INVENTION

This invention relates to compositions and methods for crosslinking tissue. More particularly, this invention relates to crosslixiked tissue with improved structural properties.

A variety of bioprostheses include tissue as at least a component of the prostheses. Such bioprostheses are used to repair or replace damaged or diseased organs, tissues and other structures in humans and animals. Examples of prostheses include, without limitation, prosthetic hearts, prosthetic heart valves, ligament repair materials, valve repair and replacement materials, and surgical patches

Tissue used in bioprostheses typically is chemically modified or fixed prior to use. Fixing stabilizes the tissue, especially from enzymatic degradation, and reduces the antigenicity. Bioprostheses generally are biocompatible due to prolonged contact with bodily fluids and/or tissues.

Tissues can contain a variety of extra cellular matrix materials including collagen, elastin, glycosaminoglycans (GAGs) and other proteins. During fixing or stabilization, crosslinking can occur within the same protein molecule and/or between different protein molecules of the extracellular matrix. Collagen is a naturally occurring protein that includes three polypeptide chains intertwined in a coiled helical conformation to form a collagen fibril.

Suitable crosslinkers include, for example, dialdehydes such as glyoxal and glutaraldehyde, carbodiimides and epoxies. Glutaraldehyde has been a preferred crosslinking agent in part because it can be used at an approximately physiological pH under aqueous conditions. In addition to crosslinking the tissue, glutaraldehyde can sterilize the tissue and reduce the antigenicity of the tissue.

### SUMMARY OF THE INVENTION

In a first aspect, the invention pertains to a tissue. The tissue includes linkers bonded to the tissue and a bridge molecule bonded between two or more of the linkers wherein the bridge molecule and the linkers are chemically different.

In a further aspect, the invention pertains to a method of crosslinking tissue. The method includes treating the tissue with a linker composition that includes linkers and a bridge composition that includes bridge molecules. The linkers bond to the tissue and the bridge molecules bond between two or more of the linkers.

In another aspect, the invention pertains to a method of bonding two or more linkers. The method includes adding bridge molecules, wherein the bridge molecules bond between the two or more linkers.

In a further aspect, the invention pertains to a composition that includes linkers and bridge molecules wherein the bridge molecules are bonded between two or more linkers and the bridge molecules and the linkers are chemically different.

In another aspect, the invention pertains to a tissue comprising bridge molecules. The tissue is modified tissue and the bridge molecules are bonded to two or more modified sites in the modified tissue.

In yet another aspect, the invention pertains to a method of crosslinking tissue. The method includes treating modified tissue with a bridge composition that includes bridge molecules wherein the bridges bond to two or more modified sites in the modified tissue.

### BRIEF DESCRIPTION OF THE DRAWINGS

FIG. 1 is a schematic diagram of a synthetic method for preparing triglycidyl amine (TGA).

FIG. 2 is a schematic diagram of a tissue treated with linkers and bridges.

FIG. 3 is a schematic diagram of extracellular matrix in a tissue treated with only linkers.

FIG. 4 is a schematic diagram of extracellular matrix in a tissue treated with linkers and bridges.

FIG. 5a is a schematic diagram of crosslinked tissue with oligomers of linker/bridge/linker conjugates.

FIG. 5b is a schematic diagram of crosslinked tissue with linker/bridge/linker conjugates of various sizes.

### DETAILED DESCRIPTION OF THE PREFERRED EMBODIMENTS

Improved approaches for crosslinking tissue described herein include the use of bridging molecules, referred to herein as bridges. In some embodiments, the bridges connect linkers that are bonded to the tissue. In other embodiments, the bridges connect two or more modified chemical moieties of a tissue. The approaches described herein can be used to crosslink a variety of extracellular matrix materials in tissue, including collagen, elastin, GAGs and other proteins. The approaches herein will be discussed mainly with respect to collagen fibrils. The approaches, however, are not limited to use with collagen fibrils but could be any component of tissue.

The size of the bridge molecules is preferably selected such that the linker/bridge/linker conjugates and/or the bridge molecule alone has a desired size to connect protein molecules in tissue separated by selected distances. In particular, bridge molecules are preferably selected to have an appropriate size to bond between proteins in different collagen fibrils, between elastin molecules, elastin molecules, GAG molecules and the like. Chemical crosslinking between proteins in different collagen fibrils is believed to yield the desirable stabilization of tissue due to the presence of crosslinks along with the desired softness and flexibility of tissue.

To provide the desired crosslinking, tissue can be treated with both linkers and bridges. The linker compound, i.e., linkers, include at least two functional groups in the linker molecule. A first functional group can chemically bond to the tissue, and a second functional group can chemically bond to a bridge. The second functional group may also be able to bond to the tissue. Chemical bonding as referred to herein refers to all types of chemical bonding including covalent bonding. In some embodiments, the first and second functional groups of the linkers are the same. The linkers may form oligomers prior to or during treatment of the tissue.

In particular, the linkers can be crosslinking agents. These crosslinking agents can be used in treating tissue resulting in covalent bonds between the crosslinking agents and the tissue. In other embodiments, the linkers include functional groups that bond with the tissue upon exposure to activators. Activators, such as certain enzymes, chemically modify the tissue to create functional groups that bond with linkers. Activators, for example, can modify the tissue by addition of aldehyde groups at particular sites in a protein molecule. The modified tissue can be treated with linkers and bridges to form crosslinked tissue. Alternatively, the modified functional groups themselves can function as linkers that directly bond with the bridges to form crosslinked tissue. The use of functional groups as linkers eliminates the complications that can result from self-polymerization of the linkers.

The bridges include at least two functional groups that can react with a functional group in the linkers in order to connect two linkers. The bridges are chemically different than linkers, and the functional groups of the bridges are generally non-reactive with unmodified tissue or with other bridges. The bridges when chemically bonded to two linkers and/or to two modified sites are appropriately sized to span the distance, for example, between collagen fibrils.

Tissues generally can be crosslinked using a variety of crosslinking agents. Monomers and/or oligomers of crosslinking agents are generally able to permeate tissue. The distance, for example, between fibrils in collagen is about the length of a molecule with about 32 carbon atoms in a covalently bonded chain. Crosslinking agent monomers are generally too small to bridge the space between proteins, for example, in different collagen fibrils. Crosslinking between proteins of the extracellular matrix are generally achieved due to self-polymerization of the crosslinking agents to an appropriate size to span the distance between proteins of the extracellular matrix.

Random self-polymerization results in a distribution of polymer sizes that bond to the tissue with only a fraction of polymers having the desired size. Other polymers/monomers are too small to span between collagen fibrils while other crosslinking polymers are excessively large. While crosslinking with excessively large crosslinking agents mechanically stabilizes the tissue, the tissue seems to lose flexibility. One method for reducing the presence of crosslinker polymers of excessive length is described in U.S. Pat. No. 5,958,669 to Ogle et al. entitled “Apparatus and Method for Crosslinking to Fix Tissue or Crosslink Molecules to Tissue”, incorporated herein by reference.

In the present approach, crosslinking moieties, i.e., linker-bridge-linker, are engineered to have a length in a desired range. In alternative embodiments, the bridges are engineered directly to have a length within a desired range with the bridges binding directly to modified functional groups in the tissue. In either case, crosslinking is enhanced to yield chemical crosslinks in the tissue that provide mechanical and chemical stabilization while yielding desired flexibility and softness of the tissue.

The methods for obtaining crosslinked tissue include treating the tissue with a bridge composition and/or a linker composition. In some embodiments, the tissue may be treated with the linker composition and the bridge composition simultaneously. In other embodiments, for example, it may be advantageous to incubate the tissue with the linker composition prior to introducing the bridge composition to the tissue. In other embodiments, the tissue may be modified using, for example, an enzyme and then treated with a bridge composition. The nature and the reactivity of the functional groups of the bridges and/or the linkers may determine the specific order of tissue treatment.

A number of bioprostheses can be used to treat patients by repairing or replacing damaged or diseased organs, tissues or other structures in humans and animals. Relevant bioprostheses are intended to contact a patient's body fluids and include a tissue component. Body fluids include, for example, blood, plasma, serum, interstitial fluids, saliva and urine. The patient can be an animal, especially a mammal, and preferably is a human. Preferably, the tissue in the bioprostheses has been treated with the bridges described herein. The tissue can be treated with the bridges either before or after being incorporated into the bioprostheses.

Tissue crosslinked using bridges can exhibit a number of advantageous properties. The crosslinked tissue is generally strong and stable while retaining a desirable amount of flexibility. In addition, the methods described herein can be used to modulate, for example, the reaction kinetics of the crosslinking and the material properties of the crosslinked tissue. For example, the concentration of the bridges, the reactivity of the bridge functional groups and the duration of the tissue treatment may determine the crosslinking kinetics and the properties of the tissue.

A. Tissue and Bioprostheses

Tissue crosslinked using the approaches described herein generally are incorporated into a medical device, generally a bioprosthesis. The bioprostheses may or may not include components other than the tissue. Appropriate bioprostheses can include, without limitation, artificial organs such as artificial hearts, ventricular assist devices, anatomical reconstruction prostheses such as jaw implants, heart valves, heart valve stents, valve leaflets, pericardial patches, surgical patches, structural stents, vascular shunts, biological conduits, pledgets, annuloplasty rings, dermal grafts for wound healing, orthopedic and spinal implants, urinary stents, permanently indwelling pericardial devices, maxial facial reconstruction plating, dental implants, intraocular lenses, bone prostheses, skin prostheses, ligament prostheses, tendon prostheses, nerve regeneration guides or tubes and combinations thereof.

Bioprostheses of particular interest include implantable vascular devices. “Vascular” sites and structures as used herein include cardiovascular sites and structures and other blood contacting sites and structures. Implantable vascular devices include, for example, vascular stents, vascular grafts and conduits, valved grafts, coronary stents, heart valves and patches.

Tissue can include natural material, synthetic material and combinations thereof. Natural tissue materials include relatively intact (cellular) tissue, decellularized and recellularized tissue. These tissues may be obtained from, for example, native heart valves; portions of native heart valves such as roots, walls and leaflets; pericardial tissues such as pericardial patches; connective tissues; bypass grafts; tendons; ligaments; skin patches; blood vessels; cartilage; dura matter; skin; bone; fascia, submucosa and umbilical tissues; and the like.

Natural tissues are derived from a particular animal species, typically mammalian, such as human, bovine, porcine, seal or kangaroo. These natural tissues generally include collagen-containing material. Natural tissue is typically, but not necessarily, soft tissue.

Appropriate tissues also include tissue equivalents such as tissue-engineered material involving a cell-repopulated matrix, which can be formed from a polymer or from a decellularized natural tissue.

Tissue, including natural tissue and tissue equivalents generally include natural proteins, such as extracellular matrix proteins. Extracellular matrix proteins include, for example, collagen and elastin. Proteins generally include molecules with one or more polypeptides and can include other non- peptide components, such as carbohydrates, lipids, nucleic acids and/or other natural or synthetic compounds, which may or may not be covalently bonded to the polypeptide.

Non-tissue components of the bioprosthesis can be formed from a variety of other biocompatible materials such as metals, ceramics and polymers. Appropriate polymers include, for example, hydrogels, reabsorbable polymers and nonreabsorbable polymers. These nontissue components can take the form of, for example, stents, cloth covers, sewing cuffs or sutures.

Appropriate synthetic polymers for use in medical devices include, without limitation, polyamides (e.g., nylon), polyesters, polystyrenes, polyacrylates, vinyl polymers (e.g., polyethylene, polytetrafluoroethylene, polypropylene and polyvinyl chloride), polycarbonates, polyurethanes, polydimethyl siloxanes, cellulose acetates, polymethyl methacrylates, ethylene vinyl acetates, polysulfones, nitrocelluloses and similar copolymers. These synthetic polymeric materials can be woven into a mesh to form a matrix or substrate. Alternatively, the synthetic polymer materials can be, for example, molded or cast into appropriate forms.

Biopolymers can be naturally occurring or produced in vitro by, for example, fermentation and the like. Purified biological polymers can be appropriately formed into fibers or yarn and then into a substrate by techniques such as weaving, knitting, casting, molding, extrusion, cellular alignment and magnetic alignment. Suitable biological polymers include, without limitation, collagen, elastin, silk, keratin, gelatin, polyamino acids, polysaccharides (e.g., cellulose and starch), nucleic acids and copolymers thereof.

B. Linkers

The linkers generally include at least two functional groups that can chemically bond with a bridge molecule. In some embodiments, the linker functional groups are the same. Alternatively, the linker can include at least two different functional groups. One or more of the functional groups in the linkers can generally bond with the tissue, and at least one functional group in the linker can generally bond with the bridges. If only one functional group of the linker reacts with the tissue, at least one other functional group reacts with the bridge molecule. Alternatively, the linkers can include one or more functional groups that react with the tissue only upon exposure of the tissue to activators.

The linkers are generally organic molecules. The linkers generally are soluble and are able to diffuse into the tissue. The linkers may include a hydrocarbon chain with appropriate functional groups. The length of the linkers is generally less than about 25 Angstroms, preferably between about 2 Angstroms and about 10 Angstroms.

In some embodiments, the linkers can be crosslinking agents. Crosslinking agents include two functional groups that bond to the tissue. The crosslinking agents can also bond to the bridges. The crosslinking agents generally covalently bond with functional groups in protein side chains. Suitable functional groups in crosslinking agents include, for example, aldehyde groups, epoxy groups, epoxyamine groups, imide groups and the like. Suitable dialdehyde crosslinking agents include, for example, glutaraldehyde, malonaldehydes, glyoxal, succinaldehyde, adipalaldehyde, phthalaldehyde and derivatives thereof. Derivatives of glutaraldehyde include, for example, 3-methylglutaraldehyde and 3-methoxy-2,4-dimethyl glutaraldehyde. Other suitable crosslinking agents include, for example, diepoxides, carbodiimide, 1-ethyl-3(3-dimethyl amino propyl)-carbodiimide hydrochloride (EDC), genipin and formaldehyde.

In some embodiments, one or more epoxyamine compounds can be used as linkers. Epoxyamines are molecules that generally include both an amine moiety (e.g. a primary, secondary, tertiary, or quaternary amine) and an epoxide moiety. The epoxyamine compound can be a monoepoxyamine compound and/or a polyepoxyamine compound. The epoxyamine compound is preferably a polyepoxyamine compound having at least two epoxide moieties and possibly three or more epoxide moieties. In one of the embodiments, the polyepoxyamine compound is triglycidyl amine (TGA).

The epoxyamine compounds are readily soluble in aqueous solutions, which is advantageous for use in the linking compositions described herein. In particular, the epoxyamine compounds can be readily solubilized without the aid of surfactants. The epoxyamine compounds may also have higher reactivity than other epoxy compounds.

Polyepoxyamine compounds can be synthesized using methods known in the art. Synthesis of epoxyamine compound is described, for example, in Ross et al., 1963 J. Org. Chem. 29:824-826, Martyanova et al., 1990, Sb. Nauch. Tr. Lenengr. In-t Kinoinzh. 2:139-141 (Chem. Abst. Nos. 116:43416 and 116:31137) and Chezlov et al., 1990, Zh. Prikl. Khim. (Leningrad) 63:1877-1878 (Chem. Abst. No. 114:121880).

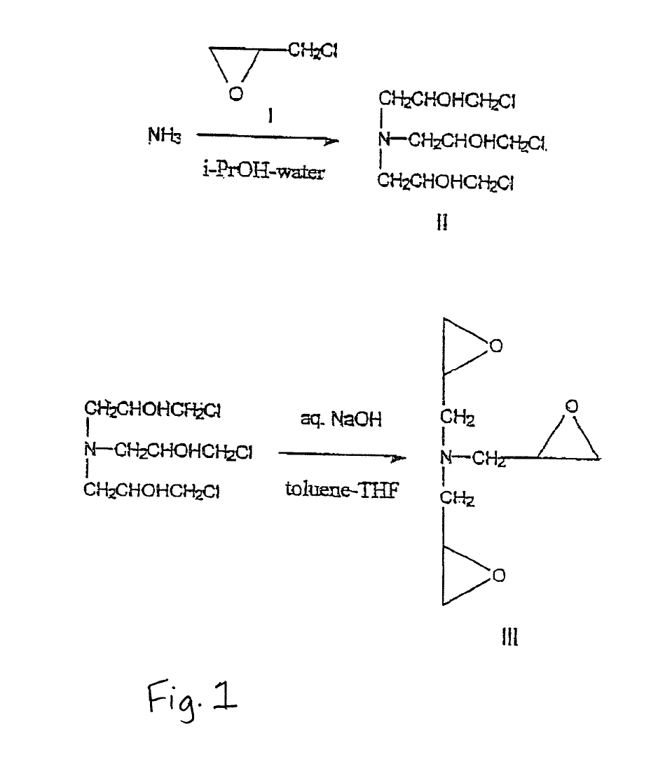

One method of synthesizing an epoxyamine is depicted in FIG. 1. Briefly, epichlorohydrin (compound I in FIG. 1) is reacted with ammonia (roughly 1:5 molar ratio with epichlorohydrin) in isopropanol with ammonium triflate as a catalyst. The reaction proceeds for about 48 hours. Following removal of volatile components, the mixture yields a viscous syrup. The syrup, after removal of unreacted epichlorhydrin with water and drying, can be dissolved in toluene and concentrated under reduced pressure to yield tris-(3-chloro-2-hydroxypropyl) amine (compound II in FIG. 1).

Compound II can be dissolved in toluene, followed by addition of tetrahydrofuran, sodium hydroxide and water. The mixture is stirred for several hours with a powerful stirrer and cooled with ice water. Then, the organic layer can be separated from the aqueous layer. The aqueous layer can be extracted with toluene and the organic phases dried overnight with a dessicant. After removing the dessicant, the solution can be concentrated under reduced pressure and the residue distilled to yield TGA (Compound III in FIG. 1). TGA can be recovered as a viscous liquid, having a boiling point of 98° C.-101° C. Liquid TGA can solidify upon refrigeration and remain a solid when returned to room temperature. The concentration of the TGA in the liquid is generally at least about 95 percent by weight or more and preferably greater than about 99 percent by weight.

In some embodiments, only one epoxyamine compound is used for crosslinking the tissue. In other embodiments, a plurality of epoxyamine compounds are used for crosslinking the tissue, such as a combination of TGA and a quaternary form of epoxyamine.

Due to the multifunctional nature of the linkers, the linkers may self-polymerize. In other words, the linkers may spontaneously form oligomers such as dimers, trimers and other higher molecular weight molecules. The polymers generally retain unreacted functional groups that can react with tissue and/or a bridge.

Preferably, linkers are reactive with the tissue at physiological temperatures and pH. Specifically, linkers preferably bond with tissues at temperatures between about 4° C. and about 37° C. Similarly, linkers preferably bond with tissue at pHs between about 4 and about 11, and more preferably at pHs between about 6 and about 9.

An aqueous solution of the linker composition may be added directly to the tissue and/or combined with a bridge composition prior to addition to the tissue. The linker composition may also include salts and/or a buffer. Suitable salts can include, for example, sodium chloride, potassium chloride and the like. Suitable buffers can be based on, for example, the following compounds: ammonium, phosphate, borate, bicarbonate, carbonate, cacodylate, citrate, and other organic buffers such as tris(hydroxymethyl) aminomethane (TRIS), morpholine propanesulphonic acid (MOPS), and N-(2-hydroxyethyl) piperazine-N′ (2-ethanesulfonic acid) (HEPES). Suitable buffers are generally chosen based on the desired pH range for the linker composition. TRIS buffers, for example, act as buffers in the pH range of between about 6 and about 8.

C. Modified Tissue

In some embodiments, the tissue can be modified by activators prior to treatment with the bridges. Activators interact with a protein or other matrix material to modify functional groups within the tissue. The modified sites can include, for example, an aldehyde group. The activators can generally modify a number of sites within the tissue. A variety of activators can be used to modify proteins and include, for example, enzymes, ultraviolet light, visible light, dye with ultraviolet light and the like. Lysyl oxidase, for example, can activate a protein by creating an aldehyde functional group on the protein. Other suitable enzymes that modify tissue can include, for example, mono-functional enzymes such as lysyl oxidase, transglutaminase, peroxidase, xanthine oxidase and the like. In such instances, the modified protein, and more particularly the aldehyde group added to the modified protein can act as a linker.

D. Bridges

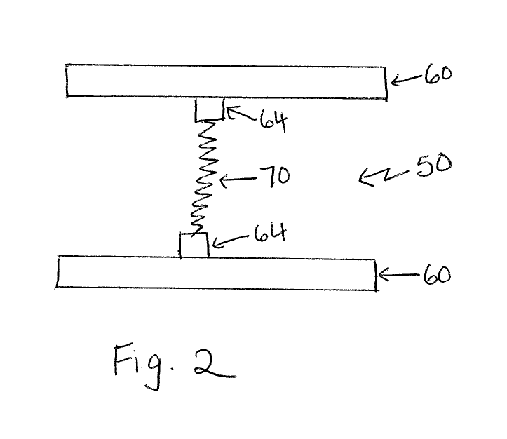

Bridges are chemically distinct from the linkers. Bridges have two or more functional groups and chemically bond with two or more linkers. Bridges are generally non-reactive with respect to unmodified tissue but reactive with linker functional groups such as aldehydes, epoxies and the like. In an illustrative embodiment shown in FIG. 2, bridge 70 connects two linkers 64. Linkers 64 are bonded to extracellular compounds 60 in tissue 50. In addition, bridges can be chemically reactive with the modified tissue described herein. By reacting to the linkers or modified tissue, the bridges chemically crosslink the tissue. In preferred embodiments, the bridges are selected based on their solubility and the desired length to form crosslinks.

Bridges generally include at least two or more functional groups. The functional groups may be equivalent or different. Suitable functional groups on the bridge molecules react with the linkers or modified tissue and include, for example, methylthio groups, thio groups, amine groups, alcohol groups, carboxyl groups and the like. Preferred functional groups on the bridge molecules include amine groups and thio groups. The functional groups of the bridges are preferably at opposite ends of the bridge molecule. The functional groups of the bridges, preferably, react with the linkers and modified tissue without additional catalysts. However, the functional groups of the bridges may react with the linkers and modified tissue with addition of catalysts.

Bridges generally include a hydrocarbon backbone. Suitable molecules for use as bridges can include molecules that have chains or rings with spaced apart functional groups sufficient to span the distance between extracellular matrix materials, i.e. collagen fibrils, when bonded to two molecules of linkers or modified functional groups in tissue proteins. When flexibility of the crosslinked tissue is desired, the bridges include a saturated hydrocarbon backbone without any rings. Alternatively, the rigidity of the crosslinked tissue can be increased, if desired, by the addition of rings and unsaturated bonds to the bridge. Increased rigidity is desirable, for example, in prostheses to replace bone and/or cartilage.

Suitable bridges for linkers or modified tissue that include aldehydes and genipin, include, for example, 1,3-diaminopropane, 1,4-diaminobutane, 1,5-diaminopentane, 1,6-diaminohexane, 1,7-diaminoheptane, 1,8-diaminooctane, 1,9-diaminononane, 1,10-diaminodecane and the like.

Suitable bridges for linkers such as EDC include, for example, 1,3-dicarboxylpropane, 1,4-dicarboxylbutane, 1,5-dicarboxylpentane, 1,6-dicarboxylhexane, 1,7-dicarboxylheptane, 1,8-dicarboxyloctane, 1,9-dicarboxylnonane, 1,10-dicarboxyldecane and the like.

Suitable bridges, for linkers that include epoxies and epoxyamine, include, for example, 1,3-propanedithiol, 1,4-butanedithiol, 1,5-pentanedithiol, 1,6-hexanedithiol, 1,7-heptanedithiol, 1,8-octanedithiol, 1,9-nonanedithiol, 1,10-decanedithiol, 1,3-diaminopropane, 1,4-diaminobutane, 1, 5-diaminopentane, 1, 6-diaminohexane, 1,7- diaminoheptane, 1,8-diaminooctane, 1,9-diaminononane, 1,10-diaminodecane, 1,3-propanediol, 1,4-butanediol, 1,5-pentanediol, 1,6-hexanediol, 1,7-heptanediol, 1,8-octanediol, 1,9-nonanediol, 1,10-decanediol and the like.

Suitable bridges can also be short, diffusible fragments of extracellular matrix, for example, collagen, GAGs, a fibrillar and the like.

Some bridges can have functional groups that bond to modified tissue upon exposure to visible light in the presence of a photocatalyst. Photocatalysts can be dyes, for example, methylene green, methylene blue, rose bengal, riboflavin, proflavin, fluorescein and the like. Dyes can react with the modified tissue, for example, aldehyde groups on modified tissue. Bridges that react with tissue upon exposure to photocatalysts include, for example, bifunctional amines.

The bridges can be a variety of sizes. The bridges generally are of appropriate size to diffuse into tissue. In addition, the bridges generally are soluble in aqueous solutions. The size of the bridges selected may depend on the size of the linker used. The linker/bridge/linker conjugate is generally sufficiently sized to span the distance between proteins in the extracellular matrix, for example, the distance between collagen fibrils. Suitable bridges may have a hydrocarbon backbone that includes between about 4 Angstroms and about 15 Angstroms. The bridges may also include branches in the hydrocarbon backbone, thus increasing the number of carbons but not necessarily the span of the bridge. Bridges with hydrocarbon backbone of more than 10 carbons can be used, for example, if the bridge includes carboxyl group functionalities.

Preferably bridges are generally reactive with the linkers at physiological temperatures and pH. Bridges are preferably reactive at temperatures between about 4° C. and about 37° C. Bridges are preferably reactive at pHs between about 4 and about 11 and more preferably between pH of about 6 and about 8.

An aqueous bridge composition can also include salts and/or a buffering system. Suitable buffering systems to be used in bridge compositions are as described above for linker compositions.

E. Crosslinked Tissue

The crosslinked tissue described herein includes a plurality of bridges bonded to the tissue either through linkers or through modified sites in tissue. In particular, the bridges can connect linkers or modified sites in separate proteins, such as proteins on different collagen fibrils.

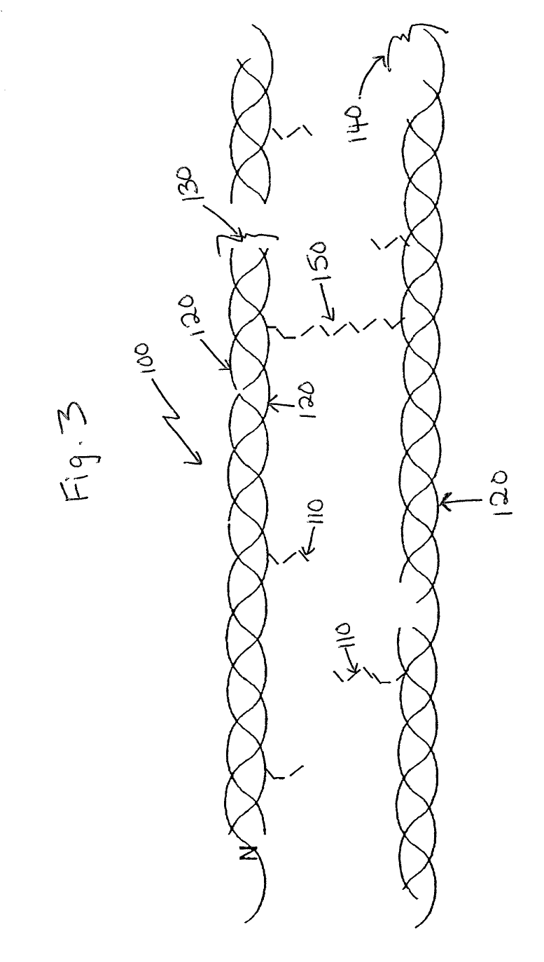

FIG. 3 illustrates a crosslinked tissue 100 with the use of only linkers 110 but without the use of bridges. Linkers 110 bond with a polypeptide 120 of collagen fiber 130. Oligomers of the linkers can be attached to the tissue at multiple sites. A portion of the linkers self-polymerize sufficiently to form crosslinks 150 and connect tissue 100 from fiber 130 to fiber 140 as indicated at site 150.

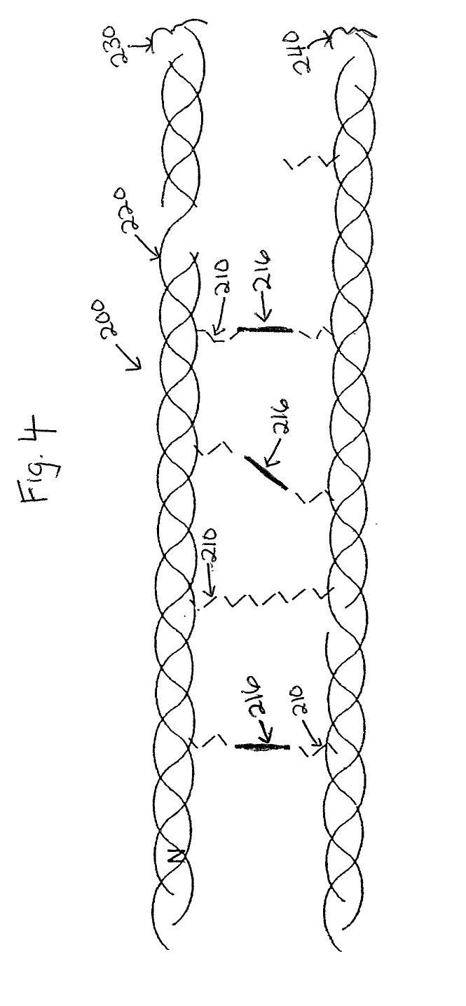

FIG. 4 is an illustrative embodimenast of crosslinked tissue 200 treated using linkers 210 and bridges 216. Linkers 210 bond to polypetides 220 of collagen fiber 230. Oligomers of the linkers can be attached to tissue 200 at multiple sites. Bridges 216 can bond to two molecules of linkers 210 to connect tissue 200 from fiber 230 to fiber 240. The use of bridges 216 can increase the amount of connections or crosslinks between fibers 230 and 240. Bridges can connect two linkers that normally may not reach each other or other sections of the tissue.

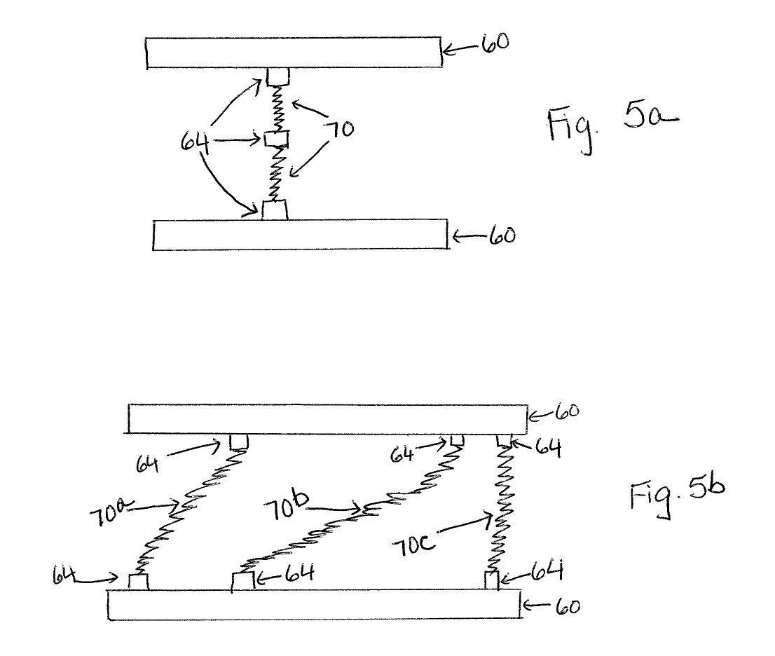

The linker/bridge/linker conjugates in the crosslinked tissue generally span a distance of at least about 10 Angstroms, preferably between about 15 Angstroms and about 100 Angstroms, and more preferably between about 25 Angstroms and about 50 Angstroms. Smaller linker/bridge/linker conjugates may be suitable because the conjugates may include additional linkers and/or bridges, for example, as shown in FIG. 5a. In FIG. 5a, tissue 60 is crosslinked with three molecules of linkers 64 connected by two bridges 70. In other words, linker/bridge/linker conjugates may oligomerize to form a sufficiently large conjugate to span the desired distance. Similarly, a single bridge can bond to linker oligomers to form a linker oligomer/bridge/linker conjugate. Other combinations of structures with oligomers can similarly form. Suitable linker/bridge/linker conjugates can also vary in size because larger conjugates that span a greater distance may be suitable to connect sites on the proteins that are relatively far away as shown in FIG. 5b. In FIG. 5b, tissue 60 is crosslinked with linkers 64 and bridges 70a, 70b and 70c of varying size.

The crosslinked tissue can include bridges that connect linkers bonded to different fibrils of collagen. The crosslinked tissue can include bridges that connect modified sites on different fibrils of collagen. The bridges in the crosslinked tissue may also connect linkers or modified sites that are on different polypeptides in the same collagen fibril. The bridges in the crosslinked tissue may also connect modified sites and/or linkers bonded to a single polypeptide. The bridges, however, can be particularly suited for connecting modified sites in tissue or linkers that are on different proteins of the extracellular matrix materials.

The crosslinked tissue including the bridges can be advantageously flexible and strong. Crosslinked tissue can be evaluated by one or more of several established criteria such as thermal stability (i.e. shrink temperature), digestibility by enzymes, amino acid analysis and mechanical properties such as extensibility, elasticity and tensile strength. Additionally, the character of crosslinked tissue can be further evaluated through both in vivo and in vitro biocompatibility assessment. Desirable properties can vary depending on the specific application of the crosslinked tissue.

F. Methods of Crosslinking Tissue

The improved methods of Crosslinking Tissue can involve treating the tissue with bridges. In some embodiments, the tissue is crosslinked by treating the tissue with both linkers, particularly crosslinking agents, and bridges to obtain crosslinked tissue. In other embodiments, the tissue is modified by an activator to generate modified protein functional groups which can in turn form bonds with the bridges.

The tissue can be treated by the linker composition and the bridge composition simultaneously. Alternatively, the tissue may be treated sequentially with the linker composition and the bridge composition. In other embodiments, the linkers and bridges are incubated together to form linker/bridge conjugates prior to addition to the tissue. The linker/bridge conjugates can include two molecules of linkers at either end and a bridge molecule connecting two molecules of the linkers. The desired conjugate may be selected by, for example, screening based on molecular weight. Screening for molecules with the desired size can be performed as described in U.S. Pat. No. 5,958,669 to Ogle et al., incorporated herein by reference. When the tissue is treated with the conjugates, the linker molecules at either end of the conjugates may then react with the tissue.

The method of crosslinking the tissue may also include exposing the tissue to ultraviolet light for photocoupling. The tissue and the linking compounds can be photocoupled for covalent bonding, for example, by using high energy light, such as ultraviolet light, to form reactive intermediates of the functional groups on the linkers. The reactive intermediates can form carbon-carbon bonds between the linkers and tissue. Aryl ketone groups are particularly useful in this respect.

Photochemical coupling can also be used for attaching bridges to the linker. See, for example, Dunkirk et al., J. Biomaterials Applications 6:131-156 (1991), incorporated herein by reference. The bridges may or may not be present when the tissue is exposed to the ultraviolet light. The bridges may be added after the tissue with the linkers has been exposed to the ultraviolet light.

The method of crosslinking the tissue may also include treating the tissue with activators to form modified tissue. Modified tissue, in turn, can form bonds between bridges and the modified sites, i.e., newly generated functional groups in the modified tissue. The tissue can be incubated with, for example, enzymes, particularly mono-functional oxidases such as lysyl oxidase, to generate an aldehyde group on the protein. The proteins with added aldehyde groups can bond with bridges, for example, bridges with multiple amino functional groups, at the modified sites.

In embodiments using enzyme activators, the desired enzyme may be added to an aqueous solution along with the bridges. Alternatively, the tissue can be treated first with the enzymes to form the modified functional groups. Bridges can then be added to the modified tissue to bond with the modified functional groups. By adding the activators, such as enzymes, and bridges sequentially, the specific conditions in which the bonding of the bridges to the modified tissue is conducted may be different than the conditions in which the activators modify the tissue.

The method of crosslinking the tissue may also include exposing the tissue to visible light. Visible light can include, for example, incandescent light, white light, fluorescent light and other visible light absorbed by any photocatalyst present. Generally, in order to bond bridges to tissue using visible light, the tissue is exposed to a photocatalyst that can mediate the bond formation. A photocatalyst, for example, can be included in the bridge composition. Photocatalysts, when activated by light, generally transfer electrons or hydrogens atoms, and thereby oxidize a substrate in the presence of oxygen. Dyes, for example, can be used as photocatalysts and dye-mediated photooxidation is described, for example, in U.S. Pat. No. 5,147,514 to Mechanic entitled “Process for Crosslinking Collagenous Material and Resulting Product,” incorporated herein by reference. Exemplary dyes include, for example, methylene blue, methylene green, rose bengal, riboflavin, proflavin, fluorescein, eosin and pyridoxal-5-phosphate.

Preferred combinations of linker composition and bridge composition include, for example, triglycidyl amine linkers with bridges having dithiol groups, diamino groups and/or diol groups. Preferred combinations can also include glutaraldehyde as the linker with bridges having diamino functional groups.

The linker and/or bridge compositions may also include modifying agents for specific purposes, such as metals, preferably transition metals such as iron, that can accelerate enzymes, if present. The linker and/or bridge compositions may include anti-calcification agents such as osteopontin. The linker and/or bridge compositions may also include growth factors such as VEGF. Antimicrobial agents may also be included in order to prevent microbial colonization. The modifying agents may be applied separately to the tissue or they may be included in the linker and/or bridge compositions.

The tissue can be treated with the linker and/or bridge compositions for varying lengths of time. The length of the treatment may depend on the specific linker used. The tissue is generally treated for at least about 10 minutes. The tissue is preferably treated between about 1 hour and about 1 month and more preferably between about 8 hours and about 96 hours. The appropriate period of time can be determined based on mechanical strength, shrink temperature and/or amino acid analysis.

The concentration of the aqueous solutions with the linkers and/or the bridges, respectively, used to treat the tissue can vary depending on the reactivity of the specific compounds used. Generally, the concentration of the linkers in the linker composition is between about 0.0001 molar and about 1.0 molar, preferably between about 0.001 molar and about 0.7 molar, and even more preferably between about 0.01 molar and about 0.5 molar.

The concentration of the bridges in the bridge composition is generally between about 1×10⁻⁷ molar and about 1 molar, preferably, between about 1×10⁻⁵ molar and about 0.8 molar, and more preferably, between about 1×10⁻⁵ molar and about 0.5 molar.

The ratio between the amount of linkers used and the amount of bridges used can vary depending on the specific protocol used for crosslinking. When the tissue is treated with the linkers and the bridges simultaneously, the ratio of the linkers to bridges can be lower than when the tissue is sequentially incubated with linkers and then bridges.

The use of the bridges, and in some embodiments in conjunction with linkers, can allow modulation of the character of crosslinking in tissue and, thus, the properties of the crosslinked tissue. The use of a bridge composition can increase the number of crosslinks that are formed over large distances, for example, between extracellular matrix, as shown in FIG. 4. The use of the bridge composition can also result in more uniform crosslinking. Generally, the bonds between the bridges and the linkers and/or modified sites in modified tissue are readily formed. In particular, self-polymerization of the linkers is not required. Monomers and/or oligomers of linkers bonded to different fibrils or modified sites on different fibrils can, thus, be readily connected by the bridges to form fibril to fibril crosslinks.

Increasing the concentration of the bridges in the bridge composition used to treat the tissue, preferably relative to the linker concentration, for example, can increase the number of crosslinks formed, via the bridges, between the molecules of linkers. Similarly, decreasing the concentration of the bridges used in treating the tissue can decrease the number of bridge connections between linkers. The number of crosslinks in the tissue can, thus, be modulated by adjusting the concentration of the bridges and/or the linkers used for treating the tissue. Increase in the number of bridges incorporated into the tissue may decrease the flexibility of the tissue. The desired flexibility of the tissue, thus, can influence the concentration of the bridges used in the modifying composition.

The amount of crosslinking in the tissue can also be adjusted by modulating the reaction kinetics between the tissue, the linkers and the bridges. Exposure time of the tissue to the bridge composition and/or the linker composition can be varied depending on the degree of modification, i.e. crosslinking, desired. The tissue may be treated with the bridge composition for a longer time to obtain tissue with a high degree of crosslinking. Alternatively, tissue may be treated with the linkers and the bridges for a short period of time to obtain the desirable degree of crosslinking.

Using the linker and/or the bridge compositions, crosslinked tissue can be obtained with the desired material properties. The concentration of the linkers and bridges and/or the reaction time of the treatment of the tissue can be selected to obtain the desirable strength, stability and flexibility of the crosslinked tissue. If tissue with greater strength is desired, for example, the concentration of the bridges and/or the time of the tissue treatment may be increased to obtain the desired material properties such as strength. Increased crosslinking can also result in resistant to degradation.

The crosslinked tissue with the linkers and bridges can be incorporated into bioprostheses. The crosslinked tissue can form an entire bioprosthesis by itself or the crosslinked tissue can be incorporated with other biocompatible components into a bioprosthesis. Heart valve prostheses preferably include the crosslinked tissue crosslinked using the linkers and bridges described herein. The heart valve prosthesis, preferably, has increased strength and stability along with the desired flexibility.

The crosslinked tissue can be stored appropriately prior to or following formation into a bioprosthesis. Generally, the crosslinked tissue is stored in a moist, sterile environment. Other compounds such as an alcohol can be added to the storage solution. In addition, the tissue can be treated with anticalcification compositions or other compositions prior to storage or after storage.

The bioprosthesis comprising the tissue can be placed in a package along with packing material and appropriate labeling. Additional sterilization can take place prior to or following packaging. Radiation, chemicals and/or plasma can be used in the sterilization process. The packaged device is distributed to the appropriate medical personnel. The device incorporating the tissue preferably is rinsed in sterile saline solution prior to administration by medical personnel.

### EXAMPLES

### Example 1

Crosslinking with Glutaraldehyde and Diaminopentane

This example illustrates crosslinking of heart valve cusps or leaflets using glutaraldehyde as the linker compound and diaminopentane as the bridge molecules.

Solutions for this example were 0.9% saline solution, 0.5% citrate buffered glutaraldehyde (pH 6.4), 0.5% HEPES buffered glutaraldehyde (pH 7.2), and 5% 1,5-diaminopentane. A 0.9 percent saline solution was made by combining 9 grams of sodium chloride with 1000 ml of water. The citrate buffered glutaraldehyde solution was prepared by combining 10 ml of 50% by volume glutaraldehyde, 3.9 grams of sodium chloride, 0.5 grams of citric acid, 14.0 grams of sodium citrate to make 1 liter of solution. The HEPES buffered 0.5% glutaraldehyde solution was made combining 10 ml of 50% by volume glutaraldehyde solution, 9 gm sodium chloride and 11.9 gm of HEPES to make 1 liter of solution. The 5% 1,5-diaminopentane solution was made by adding 5.1 ml of a 98% by weight stock concentration solution to 100 ml sterile water.

Valve cusps were excised from porcine hearts and immersed in chilled saline. The beaker was covered with parafilm and placed onto a orbital shaker table maintained at 4° C. for 4 hours. During the 4 hours, the saline was changed twice.

Standard glutaraldehyde fixing was performed by placing the valve cusps in the citrate buffered glutarldehyde solution for 24 hours. The solution was exchanged with the citrate buffered glutaraldehyde solution for a period of 6 days.

Washed cusps were divided into 5 groups of 10 cusps per group. The groups were as follows:

Group 1—Pre-incubated with 5% diaminopentane overnight(12-16 hrs.), then standard glutaraldehyde fixation

Group 2—Unfixed control

Group 3—Standard glutaraldehyde fixation

Group 4—Fixed 1 minute with citrate buffered glutaraldehyde, then overnight incubation with 5% diaminopentane

Group 5—Fixed 1 minute with citrate buffered glutaraldehyde, overnight incubation with 5% diaminopentane, standard glutaraldehyde fixation for 6 days

Groups 2 and 3 were treated in separate 100 ml glass beakers. Groups 1, 4 and 5 were treated in the 12 well tissue culture plates with one plate per group and one cusp per well.

Groups 4 and 5 were fixed with citrate buffered glutaraldehyde for 1 minute. All 20 cusps were fixed together in a 100 ml beaker. Cusps were placed in the beaker first then the glutaraldehyde was added. Timing began once the glutaraldehyde was added. At the end of 1 minute the glutaraldehyde was poured off and the cusps were placed into individual wells of their designated plates along with the corresponding incubation solution. Group 1 wells were also filled with the diaminopentane incubation solution. For the groups 1, 4 and 5, approximately 2 ml of 5% diaminopentane was pipetted into each well containing a cusp. Plates were parafilmed closed and put on a shaker table in the refrigerator.

After 24 hours, solutions in wells for groups 1, 4 and 5 were pipetted off using a different disposable transfer pipet for each group. Processing of group 4 cusps was completed at this point, and saline was added to each well. Groups 1 and 5 had approximately 2 ml of citrate buffered glutaraldehyde added to each well containing a cusp. Plates were covered with parafilm and placed back on the shaker table in the refrigerator.

For groups 2 and 3, approximately 100 ml of the appropriate solution was poured into the beaker. Beakers were covered with parafilm and placed in the refrigerator on a shaker table.

After 24 hours, group 3 had the citrate buffered glutaraldehyde poured off, and the solution was replaced with approximately 100 ml of HEPES buffered glutaraldehyde. This beaker was covered with parafilm and placed back on the shaker table in the refrigerator.

Group 2 was the control and nothing more was done with those cusps. Groups 2 and 4 were complete at this point and were placed in the refrigerator but were no longer on the shaker table.

Groups 1 and 5 had solutions changed on day 3. Due to the amount of debris in the wells, cusps were put into clean plates. When the cusps were transferred, each cusp was rinsed in a beaker containing HEPES buffered glutaraldehyde to remove the excess debris. Once all cusps were in the new plates, approximately 2 ml of fresh HEPES buffered glutaraldehyde was added to each well. Separate rinse beakers were used for each group. Both plates were closed with parafilm and placed back in the refrigerator on the shaker table.

At the end of fixation all groups were tested for shrink temperature and lysine analysis, Shrink temperature analysis was performed by Differential Scan Calorimetry (DSC). DSC measures the shrink temperature (Tₛ) which is the temperature at which the collagen fibrils denature and the tissue shrinks. Uncrosslinked tissue has shrink temperatures of about 60° C.-65° C. and glutaraldehyde crosslinked tissue historically has had a shrink temperature of about 82° C. to about 90° C. For each of the groups, cusps segments were weighed wet using a Metier A201 balance. The sample shrink temperature was measured for each tissue.

Lysine analysis was conducted in tissue by high pressure liquid chromatography (HPLC). Cusp samples were rinsed with pure water and then lyophilized until dry. The samples were weighed and transferred to an individual, labeled hydrolysis vial. To each vial a 1.0 ml of 6 N HCl was added. Samples were purged of air and nitrogen was added. The set of hydrolysis vials were placed in an oven at about 150° C. for about 60-65 minutes. Samples were allowed to cool to room temperature. The contents of the vial were transferred to a labeled 10 ml volumetric flask and brought to volume with reverse osmosis filtered (RO) water. Aliquots of these hydrolysates were derivatized with AccQFluor derivatization system kit purchased from Waters in Milford, Mass. Aliquots of the above samples were injected onto a Waters HPLC system. L-lysine HCl was used to develop a calibration curve. This curve was used to determine the concentration of free lysine in the cusps and controls.

Table 1 shows the results from the shrink temperature analysis. Table 2 shows the results from the lysine content analysis.

TABLE 1

| Sample # | Group 1 | Group 2 | Group 3 | Group 4 | Group 5 |
|---|---|---|---|---|---|
| A | 82.6 | 55.9 | 85.0 | 65.0 | 84.3 |
| B | 81.8 | 63.0 | 85.4 | 64.2 | 82.8 |
| C | 83.5 | 64.4 | 85.4 | 64.4 | 84.9 |
| D | 83.1 | 63.5 | 87.5 | 62.1 | 84.1 |
| E | 82.3 | 63.7 | 88.3 | 65.4 | 84.6 |
| F | 82.0 | 62.0 | 83.5 | 65.8 | 82.8 |
| G | 83.1 | 64.6 | 84.8 | 63.0 | 88.2 |
| H | 81.7 | 63.9 | 86.5 | 62.6 | 85.2 |
| I | 80.8 | 62.7 | 84.8 | 64.3 | 84.6 |
| J | 82.1 | 63.7 | 85.1 | 63.2 | 86.8 |
| Avg. | 82.3 | 62.8 | 85.6 | 64.0 | 84.8 |
| SD | 0.8 | 2.5 | 1.4 | 1.2 | 1.6 |

Students T-test P-values:
Group 1 vs. Group 3 4.3E−06
Group 1 vs. Group 5 3.7E−04
Group 3 vs. Group 5 2.6E−01
Group 2 vs. Group 4 2.1E−01

TABLE 2

| Sample # | Group 1 | Group 2 | Group 3 | Group 4 | Group 5 |
|---|---|---|---|---|---|
| A | * | 127.1 | 35.1 | 132.1 | * |
| B | * | 145.6 | 34.7 | 158.0 | * |
| C | * | 125.1 | 32.5 | 170.0 | * |
| D | * | 144.6 | 32.9 | 93.4 | * |
| E | * | 183.8 | 31.2 | 157.9 | * |
| F | * | 178.9 | 32.4 | 99.9 | * |
| G | * | 213.0 | 41.7 | 65.2 | * |
| H | * | 149.1 | 31.0 | 135.8 | * |
| I | * | 117.0 | 29.1 | 91.8 | * |
| J | * | 125.5 | 28.0 | 153.8 | * |
| Avg. | | 151.0 | 32.9 | 125.8 | |
| SD | | 31.3 | 3.8 | 35.7 | |

Students T-test P-values:
Group 2 vs. Group 3 6.2E−10
Group 2 vs. Group 4 1.1E−01
Group 3 vs. Group 4 1.8E−07

All the fixed cusp groups exhibited no discernable differences in flexibility while being handled. Shrink temperature is a measure of tissue resistance to thermal denaturation. It has been correlated to the degree of tissue crosslinking. There were significant differences seen in many of the groups as calculated by the statistical program Xcel. Significant differences in Tₛ occurred between groups 1&2, 1&5, 2&3, 2&5, 3&4 and 4&5. Overall these studies suggest that bridges are capable of modulating tissue cross-linking.

Free lysine has long been considered a measure of the degree of crosslinking. Significant differences in lysine content were observed between groups. Some, but not all, were consistent with shrink temperature findings. Specifically, samples in groups 1 and 5 were not significantly different from group 3 in terms of Tₛ, yet were strikingly different in amino acid content. Further, groups 1 and 5 were resistant to acid hydrolysis (6M HCl for 2 hours at 150° C.). For groups 1 and 5, no lysine was measured, presumably because the tissue segments were so stable from extensive crosslinking that acid hydrolysis was ineffective to decompose the proteins into their composite amino acids.

### Example 2

Crosslinking with TGA and pentanedithiol

This example illustrates crosslinking of valve cusps using TGA as the linker and 1,5 pentanedithiol as the bridge.

Solutions used in this example were 0.9% Saline Solution as described in example 1, a 0.1M TGA solution, Borate-Mannitol Buffer, and 5% 1,5-pentanedithiol. Borate Mannitol buffer was made by combining 95.3 gm of sodium teteraborate decahydrate, 150 gm of D-mannitol to make 10 liters of the buffer at pH of 7.4. A 5% 1,5-pentanedithiol solution was made by combining 5 mls of 98% by weight 1,5-pentanedithiol purchased from ACROS Organics to make a 100 ml solution. Fresh TGA solution was made daily just before use of the solution. TGA was synthesized by Hawkins Chemical using the procedure described above. Borate-mannitol buffer is a stable solution and was able to be made up about a week prior to use. The 5% 1,5-pentanedithiol solution was prepared just prior to use.

Cusps were excised from porcine hearts and placed into a beaker with chilled saline. The beaker was covered with parafilm and placed into the refrigerator on a shaker table overnight. There was one saline change during the 24 hours.

The cusps were divided into 5 groups of 10 cusps per group. The groups were as follows:

Group 1—TGA fixed.

Group 2—TGA fixed for 24 hours, 5% pentanedithiol for 24 hours then standard TGA fixation.

Group 3—TGA fixed for 24 hours, then 5% pentanedithiol for 24 hours.

Group 4—5% pentanedithiol for 24 hours then standard TGA fixation.

Group 5—Unfixed control All fixations in this experiment were performed at room temperature.

TGA fixation was done for 24 hours with groups 1, 2 and 3. A 2.5 ml volume of TGA was pipetted into each well containing a cusp. Plates were closed with parafilm and placed on a shaker table at room temperature.

After 24 hours, the TGA was removed from the wells. Then, groups 2, 3 and 4 had 2.5 ml of 5% 1,5-pentanedithiol pipetted into each well. Plates were closed with parafilm and returned to the shaker table. Group 1 had 2.5 ml of fresh TGA pipetted into each well. The 5% 1,5-pentanedithiol was discarded after 24 hours. Group 3 was completed and saline was added to each well.

Groups 1, 2 and 4 had 2.5 ml of fresh TGA added to each well and were placed back on the shaker table. Plates were not closed with parafilm at this time. Groups 1, 2 and 4 continued to have the TGA changed on a daily basis until all groups had 7 days of TGA fixation.

All groups were submitted for shrink temp testing and lysine analysis.

Table 3 shows the results from the shrink temperature analysis. Table 4 shows the results from the lysine analysis in micromoles/liter.

TABLE 3

| Sample # | Group 1 | Group 2 | Group 3 | Group 4 | Group 5 |
|---|---|---|---|---|---|
| A | 83.1 | 85.1 | 172.8 | 81.0 | 155.9 |
| B | 80.0 | 84.4 | 74.0 | 80.7 | 63.0 |
| C | 82.2 | 82.9 | 73.7 | 83.4 | 64.4 |
| D | 82.6 | 84.9 | 73.1 | 81.2 | 63.5 |
| E | 83.4 | 81.7 | 74.4 | 82.0 | 63.7 |
| F | 82.6 | 85.1 | 74.5 | 82.1 | 62.9 |
| G | 82.3 | 83.2 | 73.4 | 82.1 | 64.5 |
| H | 83.6 | 83.4 | 73.1 | 81.7 | 63.9 |
| I | 84.8 | 82.9 | 73.1 | 83.4 | 62.7 |
| J | 82.4 | 83.9 | 72.2 | 81.5 | 63.6 |
| Avg. | 82.7 | 83.7 | 73.4 | 81.9 | 62.8 |
| SD | 1.2 | 1.1 | 0.7 | 0.9 | 2.5 |

Student T-test P-values:
Group 1 vs. Group 2 6.5E−02
Group 1 vs. Group 4 1.2E−02
Group 2 vs. Group 4 9.2E−04
Group 1 vs. Group 3 5.3E−14
Group 1 vs. Group 5 7.0E−13
Group 3 vs. Group 5 2.1E−09

TABLE 4

| Sample # | Group 1 | Group 2 | Group 3 | Group 4 | Group 5 |
|---|---|---|---|---|---|
| A | 123.5 | 28.7 | 176.9 | 63.2 | 127.1 |
| B | 92.3 | 73.4 | 127.1 | 54.0 | 145.6 |
| C | 94.9 | 17.5 | 162.4 | 53.9 | 125.1 |
| D | 68.8 | 36.4 | 118.8 | 33.5 | 144.6 |
| E | 108.3 | 15.8 | 84.4 | 62.6 | 183.8 |
| F | 73.1 | 43.0 | 133.3 | 83.6 | 178.9 |
| G | 114.6 | 69.5 | 142.5 | 47.8 | 213.0 |
| H | 104.3 | 100.4 | 145.8 | 61.0 | 149.1 |
| I | 75.8 | 25.3 | 140.6 | 44.5 | 117.0 |
| J | 158.4 | 83.5 | 128.2 | 54.4 | 125.5 |
| Average | 101.4 | 49.3 | 136.0 | 55.8 | 151.0 |
| SD | 27.1 | 30.0 | 25.0 | 13.3 | 31.3 |

Student T-test P-values:
Group 1 vs. Group 2 1.1E−03
Group 1 vs. Group 4 1.5E−04
Group 2 vs. Group 4 7.2E−01
Group 1 vs. Group 3 8.2E−03
Group 1 vs. Group 5 1.3E−03
Group 3 vs. Group 5 2.5E−01

Specifically, group 3 exhibited a higher Tₛ than fresh, but lower than groups 1, 2 and 4. There was also a significant difference between group 1 and group 5.

With respect to lysine analysis, increased stability was seen in groups 2 and 4 in which there were still particles of tissue in the hydrolysate after fixation. The results indicate that adding bridges alters the measurable crosslinking properties of the bioprosthetic tissue.

With respect to fixation using either glutaraldehyde as in Example 1 or with TGA, addition of bridges and then fixing results in slightly lower but statistically significant Tₛ for both glutaraldehyde and TGA samples, with very stable resistance to free lysine hydrolysis.

When the tissue was fixed prior to incubation with the bridges, the result appeared to indicate the presence of a less crosslinked matrix having slightly increased Tₛ and decreased free lysine. The Tₛ of only the group fixed with the TGA showed statistical significance.

When the tissue was fixed, incubated with the bridge and then fixed, Tₛ values were equal to or higher than any of the groups tested. The material also exhibited superior resistance to free lysine hydrolysis.

Overall, these studies suggested that bridges are capable of modulating tissue crosslinking. The increased resistance to acid hydrolysis may increase the durability of the material.

The embodiments described above are intended to be illustrative and not limiting. Additional embodiments are within the claims below. Although the present invention has been described with reference to preferred embodiments, workers skilled in the art will recognize that changes may be made in form and detail without departing from the spirit and scope of the invention.

What is claimed is:

1. A tissue comprising linkers bonded to the tissue and a bridge molecule bonded between two or more of the linkers, wherein the linkers and the bridges are chemically different.

2. The tissue of claim 1 wherein the tissue comprises extracellular matrix selected from the group consisting of collagenous fibrils, GAG and elastin.

3. The tissue of claim 1 wherein the two linkers and the bridge bonded between the two linkers span a distance of between about 10 Angstroms and about 100 Angstroms.

4. The tissue of claim 1 wherein the two linkers and the bridge bonded between the two linkers span a distance of between about 15 Angstroms and about 50 Angstroms.

5. The tissue of claim 1 wherein the bridge is a single molecule.

6. The tissue of claim 1 wherein the bridge is reactive with modified tissue.

7. The tissue of claim 1 wherein the bridge comprises functional groups selected from the group consisting of methylthio, thio, amine, alcohol, carboxyl and combinations thereof.

8. The tissue of claim 1 wherein the bridge comprises a hydrocarbon backbone.

9. The tissue of claim 1 wherein the linkers comprise monomers, dimers and oligomers.

10. The tissue of claim 1 wherein the linkers are active with respect to the tissue.

11. The tissue of claim 1 wherein the linkers comprise functional groups selected from the group consisting of aldehydes, epoxies, imide groups, photooxidative groups, enzymatically oxidative groups and combinations thereof.

12. The tissue of claim 1 wherein the linkers comprise crosslinking agents.

13. The tissue of claim 1 wherein the linker is selected from the group consisting of glutaraldehyde, triglycidyl amine and epoxy.

14. The tissue of claim 1 wherein a bioprosthetic device comprises the tissue.

15. The tissue of claim 14 wherein the bioprosthetic device is a heart valve prosthesis.

16. A method of crosslinking tissue comprising treating the tissue with a linker composition comprising linkers and a bridge composition comprising bridges wherein the linkers bond to the tissue and the bridges bond between two of the linkers, wherein the bridges and the linkers are chemically different.

17. The method of claim 16 wherein the tissue comprises proteins.

18. The method of claim 16 wherein the tissue is treated with the linker composition and the bridge composition simultaneously.

19. The method of claim 16 wherein the tissue is treated with the linker composition prior to addition of the bridge composition.

20. The method of claim 16 wherein the linker composition and the bridge composition are combined prior to treating the tissue.

21. The method of claim 16 wherein the linker composition comprises crosslinking agents.

22. The method of claim 16 wherein the concentration of the linkers in the linker composition is between about 0.0001 molar and about molar.

23. The method of claim 16 wherein the concentration of the bridges in the bridge composition is between about 1×10⁻⁷ molar and about 1 molar.

24. The method of claim 16 wherein the tissue is treated with the linker composition and the bridge composition for between about 10 minutes and about one month.

25. The method of claim 16 wherein the tissue is treated with the linker composition and the bridge composition for between about 10 minutes and about 2 weeks.

26. The method of claim 16 wherein the bridges comprise multiple functional groups.

27. The method of claim 16 wherein the treatment of the tissue further comprises exposing the tissue to activators.

28. The method of claim 27 wherein the activators are selected from the group consisting of ultraviolet light, visible light and enzymes.

29. A method of bonding two molecules of linkers comprising adding bridge molecules, wherein the bridge molecules bond between the two of the linkers.

30. A composition comprising linkers and bridge molecules wherein the bridge molecules are bonded between two of the linkers, wherein the bridges and the linkers are chemically different.

31. The composition of claim 30 wherein the bridges comprise functional groups selected from the group consisting of methylthio, amine, alcohol, carboxyl and combinations thereof.

32. The composition of claim 30 wherein the linkers comprise functional groups selected from the group consisting of aldehydes, epoxies, imide groups, photooxidative groups, enzymatically oxidative groups and combinations thereof.

33. The composition of claim 30 wherein the concentration of the linkers in the composition is between about 0.0001 molar and about 1 molar and the concentration of the bridges is between about 1×10⁻⁷ molar and about 1 molar.

34. A tissue comprising bridge molecules, wherein the tissue is modified tissue and the bridge molecules are bonded to two or more modified sites in the modified tissue.

35. The tissue of claim 34 wherein the modified sites comprise aldehyde groups.

36. A method of crosslinking tissue comprising treating modified tissue with a bridge composition comprising bridge molecules wherein the bridges bond to two or more modified sites in the modified tissue.

37. The method of claim 36 wherein the modified sites comprises aldehyde groups.
